(19) United States
(12) Patent Application Publication
(10) Pub. No.: US 2010/0031958 A1
(43) Pub. Date: Feb. 11, 2010

(54) RESPIRATORY MASK INTERFACE

(76) Inventor: Ruth Stewart, Carbon (CA)

Correspondence Address: JAY M. SCHLOFF, 6960 Orchard Lake Road, Suite 315, West Bloomfield, MI, 48322, US

(21) Appl. No.: 12/535,823

(22) Filed: Aug. 5, 2009

Related U.S. Application Data
(60) Provisional application No. 61/137,927, filed on Aug. 5, 2008.

(51) Int. Cl. A61M 16/06; A62B 18/08

(52) U.S. Cl. 128/203.29; 128/206.24; 128/206.22

(57) ABSTRACT

An interface for a respiratory mask includes a first flexible member and a second flexible member. The first flexible member is configured to be accommodated on a user's face. The first flexible member circumscribes one or more portions of the user's face. The second flexible member is configured complimentary to the first flexible member to circumscribe the one or more portions of the user's face. Further, the second flexible member is attached with the first flexible member to configure at least one hollow enclosure therebetween. In an embodiment, a hollow enclosure of the at least one hollow enclosure is filled with a cooling gel for cooling the user's face. The second flexible member is configured to accommodate the respiratory mask thereon.

### CROSS REFERENCE TO RELATED APPLICATIONS

The present disclosure claims priority under 35 United States Code, Section 119 on the U.S. Provisional Patent Application No. 61/137,927 filed on Aug. 5, 2008 the disclosure of which is incorporated by reference.

### FIELD OF THE DISCLOSURE

The present disclosure relates generally to respiratory masks, and, more particularly, to an interface for respiratory masks.

### BACKGROUND OF THE DISCLOSURE

Typically, a user may wear a respiratory mask when an ambient environment of the user is not suitable for breathing, or when the user has difficulty in breathing naturally. The respiratory mask may be configured to enclose a nasal area and/or a mouth area of the user and may form an air chamber therewithin. The air chamber may provide a gas to the user for inhaling.

Generally, the user may need to wear the respiratory mask for a long duration, especially in situations when the user is sleeping or in an unconscious state. Wearing the respiratory mask may cause discomfort to the user. Specifically, warm air that is exhaled by the user may increase temperature and humidity underneath the respiratory mask. This may cause sweating on an area enclosed by the respiratory mask. Further, some users may develop a skin irritation or a skin rash when the respiratory mask comes in contact with their skin due to sensitivity of their skin towards a material of the respiratory mask. Furthermore, the respiratory mask may suffer from a problem of leakage of the gas from edges of the respiratory mask, due to one or more gaps that may be formed between the edges of the respiratory mask and the user's skin.

In light of the drawbacks mentioned above, there is a need for preventing leakage of gases when a respiratory mask is worn by a user. Further, there is a need for preventing irritation or development of a skin rash on a user's face while wearing a respiratory mask. Furthermore, there is a need for cooling an area enclosed by a respiratory mask. Still further, there is a need for providing comfort to a user when using a respiratory mask.

### SUMMARY

In view of the foregoing disadvantages inherent in the prior art, the general purpose of the present disclosure is to provide an interface for a respiratory mask, configured to include all the advantages of the prior art, and to overcome the drawbacks inherent therein.

Accordingly, an object of the present disclosure is to provide an interface for a respiratory mask for preventing leakage of a gas from an area enclosed by the respiratory mask.

Another object of the present disclosure is to provide an interface for a respiratory mask for alleviating skin irritation or skin rash to a user.

Yet another object of the present disclosure is to provide an interface for a respiratory mask for cooling an area enclosed by the respiratory mask.

Still another object of the present disclosure is to provide comfort to a user when using a respiratory mask.

In light of the above objects, in one aspect of the present disclosure, an interface for a respiratory mask is provided. The interface includes a first flexible member and a second flexible member. The first flexible member is configured to be accommodated on a user's face. The first flexible member circumscribes one or more portions of the user's face. The second flexible member is configured complimentary to the first flexible member to circumscribe the one or more portions of the user's face. Further, the second flexible member is attached to the first flexible member to configure at least one hollow enclosure therebetween. A hollow enclosure of the at least one hollow enclosure is filled with a cooling gel for cooling the user's face or a padded insert for providing comfort to a user's face. The second flexible member is configured to accommodate the respiratory mask thereon.

This together with other aspects of the present disclosure, along with the various features of novelty that characterize the present disclosure, is pointed out with particularity in the claims annexed hereto and forms a part of this present disclosure. For a better understanding of the present disclosure, its operating advantages, and the specific objects attained by its uses, reference should be made to the accompanying drawing and descriptive matter in which there are illustrated exemplary embodiments of the present disclosure.

### BRIEF DESCRIPTION OF THE DRAWINGS

The advantages and features of the present disclosure will become better understood with reference to the following detailed description and claims taken in conjunction with the accompanying drawings, wherein like elements are identified with like symbols, and in which:

FIG. 1A illustrates a perspective view of an interface for a respiratory mask, in accordance with an embodiment of the present disclosure;

FIG. 1B illustrates a sectional side view of the interface along a section line A-A′ illustrated in FIG. 1A, in accordance with an embodiment of the present disclosure;

FIG. 2 illustrates a perspective view of an interface coupled with a respiratory mask and accommodated on a user's face, in accordance with an embodiment of the present disclosure; and

FIG. 3 illustrates an exploded view of components of FIG. 2, in accordance with an embodiment of the present disclosure.

Like reference numerals refer to like parts throughout the description of several views of the drawings.

### DETAILED DESCRIPTION OF THE DISCLOSURE

The exemplary embodiments described herein detail for illustrative purposes are subject to many variations in composition, structure, and design. It should be emphasized, however, that the present disclosure is not limited to a particular interface for a respiratory mask, as shown and described. It is understood that various omissions and substitutions of equivalents are contemplated as circumstances may suggest or render expedient, but these are intended to cover the application or implementation without departing from the spirit or scope of the claims of the present disclosure. Also, it is to be understood that the phraseology and terminology used herein is for the purpose of description and should not be regarded as limiting.

Unless limited otherwise, the terms “coupled,” “attached,” and variations thereof herein are used broadly and encompass direct and indirect arrangements. The terms “first,” “second,” and the like, herein do not denote any order, elevation or importance, but rather are used to distinguish placement of one element over another. The terms “a” and “an” herein do not denote a limitation of quantity, but rather denote the presence of at least one of the referenced items.

The present disclosure provides an interface for a respiratory mask, such as a Continuous Positive Airway Pressure (CPAP) mask. The interface is capable of being removably attached with the respiratory mask. The interface may provide comfort to a user when the user needs to wear the respiratory mask for a long duration. Specifically, the interface may be made of a cushioned material to prevent discomfort to the user from edges of the respiratory mask. Further, the interface may provide a cooling effect to an area that is enclosed by the respiratory mask. Further, the interface may be made of a material that may absorb moisture formed within the enclosed area. Furthermore, the interface may be made of non-allergenic soft filled washable or disposable material to reduce skin irritation, which may be caused by the respiratory mask. It will be obvious to a person skilled in the art that the interface may be worn with a variety of respiratory masks that may either cover the nasal area alone or the nasal area and the mouth area. Accordingly, the interface may be structured to cover an appropriate area of the user's face.

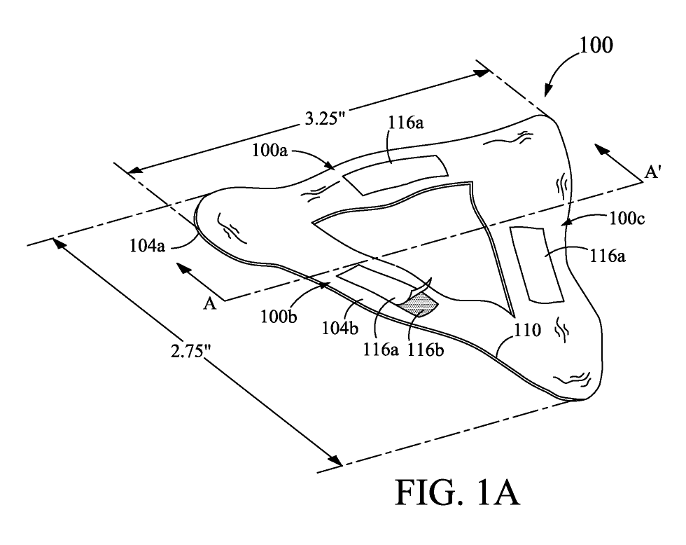

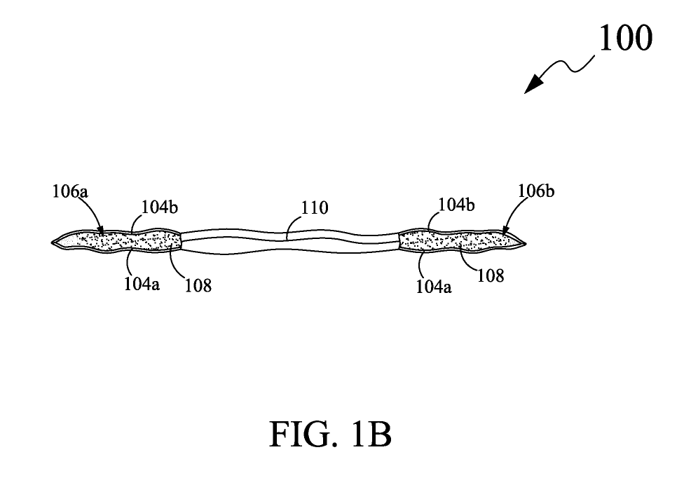

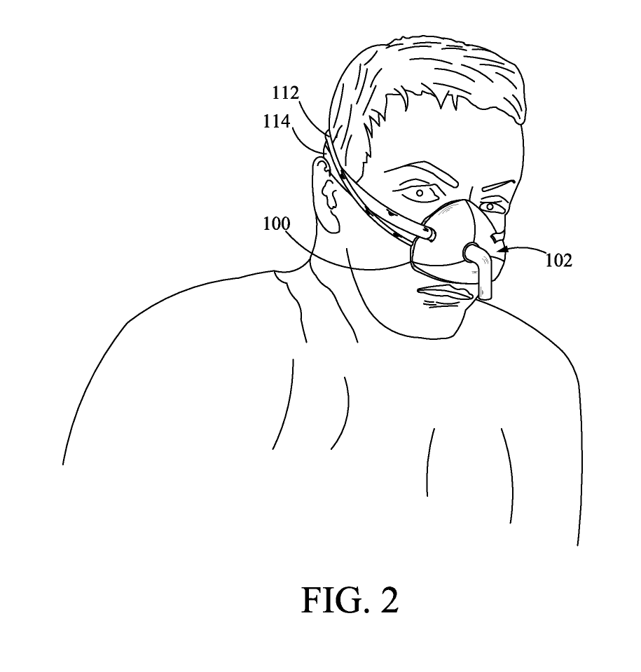

Referring to FIGS. 1A, 1B, 2 and 3, a perspective view of an interface 100, a sectional side view of the interface 100, a perspective view of the interface 100 when coupled to a respiratory mask 102, and worn by a user, and an exploded view of components of FIG. 2, respectively, are depicted. FIG. 1B illustrates the sectional side view of the interface 100 along a section line A-A′ illustrated in FIG. 1A. The interface 100 is used with the respiratory mask 102. Examples of a respiratory mask that may be coupled with the interface 100 may include a half-face mask, such as a Continuous Positive Airway Pressure (CPAP) mask, and the like. The interface 100 includes a first flexible member 104a and a second flexible member 104b (hereinafter collectively referred to as ‘flexible members 104’).

The first flexible member 104a is configured to be accommodated on the user's face. The first flexible member 104a circumscribes one or more portions of the user's face. The second flexible member 104b is configured complimentary to the first flexible member 104a such that the second flexible member 104b circumscribes the one or more portions of the user's face. The second flexible member 104b is attached with the first flexible member 104a to configure at least one hollow enclosure, such as a hollow enclosure 106a and a hollow enclosure 106b (depicted in FIG. 1B) therebetween. In an embodiment of the present disclosure, the hollow enclosure 106a and the hollow enclosure 106b are filled with a cooling gel 108 (depicted in FIG. 1B) for cooling the user's face. In an embodiment of the present disclosure, one of the hollow enclosure 106a and the hollow enclosure 106b may be filled with the cooling gel 108 such that the interface 100 provides a cooling effect to specific portions of the user's face. The second flexible member 104b is configured to accommodate the respiratory mask 102 thereon.

In another embodiment of the present disclosure, the hollow enclosure 106a and the hollow enclosure 106b may be filled with a padded insert, such that the interface 100 provides additional cushioning to a user's face.

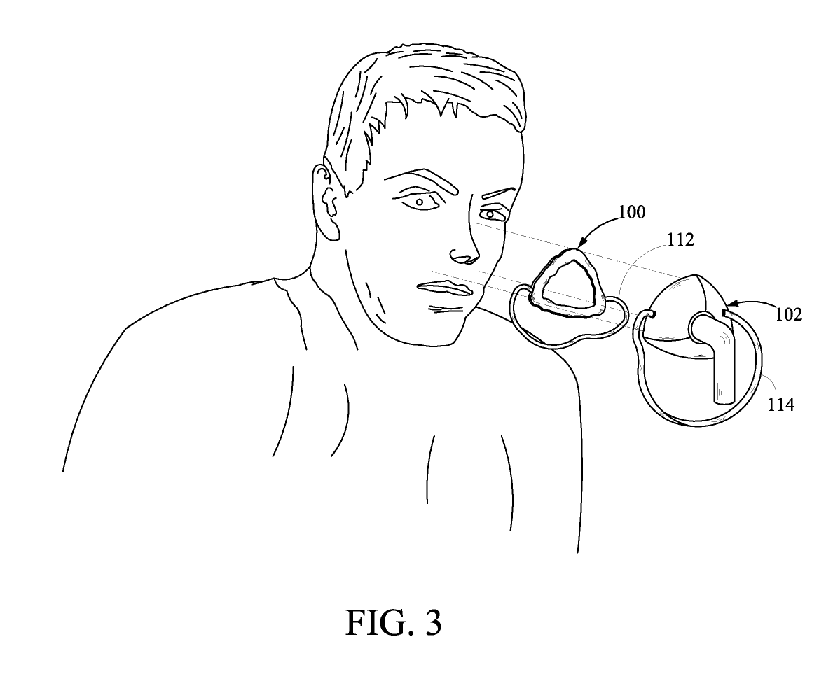

When the interface 100 is worn by the user, (as shown in FIG. 2) the first flexible member 104a contacts the user's face. The one or more portions circumscribed by the first flexible member 104a and the second flexible member 104b may include at least one of a nasal area of the user's face and a mouth area of the user's face. In an embodiment of the present disclosure, the first flexible member 104a and the second flexible member 104b may circumscribe the nasal area of the user's face. For example, the first flexible member 104a and the second flexible member 104b may be configured to be triangular in shape with a cut-off portion for accommodating the nasal area of the user's face (as depicted in FIG. 3).

In another embodiment of the present disclosure, the first flexible member 104a and the second flexible member 104b may circumscribe the nasal area and the mouth area of the user's face.

The first flexible member 104a is configured to physically contact the user's face. Accordingly, at least one of the first flexible member 104a and the second flexible member 104b may be composed of a soft material to provide comfort to the user's face. Examples of the soft material may include cotton, paper towel, and other similar materials. It may be apparent to a person skilled in the art that various other materials known in the art may be utilized without departing from the scope of the disclosure. In an embodiment of the present disclosure, at least one of the first flexible member 104a and the second flexible member 104b may be composed of a cushioning material to provide a cushioning effect to the user's face. Examples of the cushioning material may include sponge, fleece and the like. However, it may be apparent to a person skilled in the art that various other cushioning materials, known in the art, may be utilized without departing from the scope of the disclosure. Further, the cushioning material may be provided at various portions, such as a portion 100a, a portion 100b and a portion 100c of the interface 100 (depicted in FIG. 1A). Furthermore, a degree of padding of a portion of the interface 100 with the cushioning material may vary. For example, the portions 100a and 100b of the interface 100 may be configured to be positioned above the nasal area and may be provided with a higher degree of padding as compared to the portion 100c of the interface 100.

As explained previously, the flexible members 104 are configured to be complimentary in shape. For example, the flexible members 104 may be configured to be substantially triangular in shape, as depicted in FIG. 1A. In another example, the flexible members 104 may be configured to be substantially circular in shape. It will be apparent to a person skilled in the art that the flexible members 104 may be configured with various shapes, such as elliptical, square, rectangular, and the like, based on the one or more portions of the user's face to be circumscribed, without departing from the spirit and the scope of the disclosure.

Referring to FIG. 1B, the second flexible member 104b is attached with the first flexible member 104a to configure the hollow enclosure 106a and the hollow enclosure 106b therebetween, as illustrated. The second flexible member 104b may be attached to the first flexible member 104a through stitches, adhesive glue, and the like, to form an edge 110 (depicted in FIGS. 1A and 1B). However, it will be apparent to a person skilled in the art that various other techniques to attach the second flexible member 104b with the first flexible member 104a may be embodied without departing from the spirit and scope of the present disclosure. The hollow enclosure 106a and the hollow enclosure 106b are filled with the cooling gel 108 for cooling the user's face when the user's face comes in contact with the interface 100. Alternatively, one of the hollow enclosure 106a and the hollow enclosure 106b may be filled with the cooling gel 108 such that the interface 100 may provide the cooling effect to specific portions of the user's face. In another embodiment, at least one of the hollow enclosure 106a and hollow enclosure 106b may be filled with a padded insert. In an embodiment of the present disclosure, the first flexible member 104a may include a plurality of micro-perforations thereon. The plurality of micro-perforations may facilitate the cooling gel 108 to reach the user's skin. In an embodiment of the present disclosure, the interface 100 may be stored in a refrigerator or freezer to cool the cooling gel 108 prior to a refreshing application. In an embodiment of the present disclosure, the cooling gel 108 may include menthol and aloe vera to provide the cooling effect to the user's skin. However, it may be apparent to a person that the cooling gel 108 may be composed of various other materials without departing from the spirit and scope of the present disclosure.

In an embodiment of the present disclosure, the flexible members 104 may be composed of an absorbing material for absorbing moisture that may be formed on the user's face. Examples of the absorbing material may include cotton, paper towel, and other similar absorbing materials. In another embodiment of the present disclosure, the flexible members 104 may be composed of a non-allergenic material for reducing skin irritation and skin rash. In yet another embodiment of the present disclosure, the flexible members 104 may be composed of a disposable material. For example, the flexible members 104 of the interface 100 may be composed of soft paper towel or tissue material. In another embodiment of the present disclosure, the flexible members 104 of the interface 100 may be composed of a washable material. In yet another embodiment of the present disclosure, the flexible members 104 may be composed of a waterproof, moisture-breathable and hypoallergenic fabric, such as biological cotton. However, it may be apparent to a person skilled in the art that the flexible members 104 may be composed of various other materials or fabrics without departing from the spirit and scope of the present disclosure.

When worn by the user, the interface 100 circumscribes the one or more portions of the user's face. The respiratory mask 102 (shown in FIG. 3) is placed over the interface 100. In an embodiment of the present disclosure, the interface 100 may include a first coupling mechanism for coupling the interface 100 with the user's face. In one embodiment of the present disclosure, the first coupling mechanism includes one or more elastic strings, such as an elastic string 112, attached to the interface 100. The elastic string 112 is capable of being stretched to surround a portion of the user's head, thereby coupling the interface 100 with the user's face. The elastic string 112 may be attached to the interface 100 through stitches, adhesive glue, and the like. It will be apparent to a person skilled in the art that various other techniques to attach the one or more elastic strings with the interface 100 may exist, without departing from the spirit and scope of the present disclosure.

Further, in an embodiment of the present disclosure, the interface 100 may include a second coupling mechanism to couple the interface 100 with the respiratory mask 102. The second coupling mechanism may include a peel-off cover 116a and an adhesive layer 116b. The adhesive layer 116b is disposed on the second flexible member 104b and the peel-off cover 116a is removably coupled to the adhesive layer 116b. Upon removal of the peel-off cover 116a, the adhesive layer 116b facilitates coupling between the interface 100 and the respiratory mask 102. The peel-off cover 116a is composed of a non-adhesive material and is configured to cover the adhesive layer 116b. When the interface 100 is required to be coupled to the respiratory mask 102, the peel-off cover 116a may be peeled off, and the respiratory mask 102 may be placed on the interface 100. The adhesive layer 116b is configured to form a bond between the interface 100 and the respiratory mask 102.

The respiratory mask 102 may include an elastic string 114 or straps (not shown) that may be stretched to surround a portion of the user's head, thereby accommodating the respiratory mask 102 on the interface 100 and coupling the respiratory mask 102 with the user's face. When coupled to the interface 100 through the elastic string 114 (or straps), edges (not shown) of the respiratory mask 102 exert a pressure on the interface 100. Due to flexibility and cushioning effect of the flexible members 104, the pressure applied by the edges of the respiratory mask 102 fill one or more gaps that may be formed between the respiratory mask 102 and the user's skin. Filling of the one or more gaps seals an area enclosed by the respiratory mask 102, thereby preventing air from leaking from the area. Examples of the area enclosed by the respiratory mask 102 may include the nasal area of the user's face, the nasal area and the mouth area of the user's face, and the like. It may be apparent to a person skilled in the art that the nasal area will always be enclosed by the respiratory mask 102. As described above, the interface 100 may be utilized for different respiratory masks. Accordingly, the interface 100 may be produced in various sizes to accommodate the different respiratory masks thereon.

In an embodiment of the present disclosure, the interface 100 may be coupled with the user's face and the respiratory mask 102 through the elastic string 114 of the respiratory mask. Specifically, the interface 100, positioned between the respiratory mask 102 and the user's face, may be held together with the elastic string 114. The edges of the respiratory mask 102 may exert a pressure on the interface 100 while being coupled to the user's face by the elastic string 114.

As explained previously, the interface 100 may circumscribe the one or more portions of the user's face. Accordingly, the interface 100 may be configured with various shapes and may include portions of various shapes cut-off to accommodate the nasal area and the mouth area of the user. In an embodiment of the present disclosure, dimension of one side of the interface 100 that is substantially triangular may be 2.75 inches. Further, a dimension of another side of the interface 100 may be 3.25 inches. It will be apparent to a person skilled in the art that dimensions of the interface 100 may vary based on a shape of the interface 100.

The interface 100 has been previously explained to be particularly useful with the CPAP mask. However, the interface 100 may be utilized with any facial mask, particularly the facial masks that have a tendency to heat up the user's face and are uncomfortable for the user to wear. Moreover, the interface 100 may be utilized as a standalone item, without a respiratory mask. Furthermore, the interface 100 may be utilized to provide a cooling effect or cushioning effect to a portion of the user's face after a surgical operation, an injury or a skin burn. The shape of the interface 100 may be changed to fit an area of the injury or of the skin burn.

As disclosed earlier, the interface 100 may surround the nasal area of the user's face. In an embodiment of the present disclosure, an aroma-dispersing substance may be disposed on the interface 100 for dispersing at least one of aromatic scents and medicinal vapors. The aroma-dispersing substance may provide a pleasant odor or medicinal vapors, which have medicinal utility, to the user. The interface 100 may include a lavender scent to provide a pleasant aroma to the user. In another embodiment of the present disclosure, the aroma-dispersing substance may include eucalyptus vapors for providing relief from nasal congestion and other flu like symptoms.

An interface, such as the interface 100 for a respiratory mask, such as the respiratory mask 102, is advantageous for preventing leakage of a gas from an area enclosed by the respiratory mask. Further, a cooling gel, such as the cooling gel 108 in the interface may cool the user's face. Furthermore, the interface may be made of an absorbing material and may absorb moisture formed in the area enclosed by the respiratory mask, thereby reducing accumulation of the moisture. Still further, a non-allergic material of the interface may reduce skin allergies and skin irritation that may be caused to the user. Moreover, the interface may be composed of a disposable material and may be disposed of after one or more uses. Still further, a cushioning present in the interface may provide comfort to the user in wearing the respiratory mask for long hours.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present disclosure.

What is claimed is:

1. An interface for a respiratory mask, the interface comprising:
a first flexible member is configured to be accommodated on a user's face, the first flexible member circumscribing one or more portions of the user's face;
a second flexible member configured complimentary to the first flexible member to circumscribe the one or more portions of the user's face, the second flexible member attached with the first flexible member to configure at least one hollow enclosure therebetween, a hollow enclosure of the at least one hollow enclosure filled with a cooling gel for cooling the user's face,
wherein the second flexible member is configured to accommodate the respiratory mask thereon.

2. The interface of claim 1, wherein the first flexible member and the second flexible member are composed of an absorbing material for absorbing moisture formed on the user's face.

3. The interface of claim 1, wherein the first flexible member and the second flexible member are composed of a non-allergenic material.

4. The interface of claim 1, wherein the first flexible member and the second flexible member are composed of one of a washable material and a disposable material.

5. The interface of claim 1, wherein at least one of the first flexible member and the second flexible member is composed of a soft material.

6. The interface of claim 1, wherein at least one of the first flexible member and the second flexible member is composed of a cushioning material.

7. The interface of claim 1, wherein the interface further comprises a first coupling mechanism for coupling the interface with the user's face.

8. The interface of claim 7, wherein the first coupling mechanism comprises one or more elastic strings, the one or more elastic strings attached to the interface.

9. The interface of claim 1, wherein the interface further comprises a second coupling mechanism for coupling the interface with the respiratory mask.

10. The interface of claim 1, wherein the second coupling mechanism comprises a peel-off cover and an adhesive layer, wherein the adhesive layer is disposed on the second flexible member and the peel-off cover is removably coupled to the adhesive layer.

11. The interface of claim 1, wherein the one or more portions circumscribed by the first flexible member and the second flexible member comprise at least one of a nasal area of the user's face and a mouth area of the user's face.

12. The interface of claim 1, wherein an aroma-dispersing substance is disposed on the interface for dispersing at least one of aromatic scents and medicinal vapors.

13. An interface for a respiratory mask, the interface comprising:
a first flexible member is configured to be accommodated on a user's face, the first flexible member circumscribing one or more portions of the user's face;
a second flexible member configured complimentary to the first flexible member to circumscribe the one or more portions of the user's face, the second flexible member attached with the first flexible member to configure at least one hollow enclosure therebetween, a hollow enclosure of the at least one hollow enclosure filled with a padded insert,
wherein the second flexible member is configured to accommodate the respiratory mask thereon.

14. The interface of claim 13, wherein the first flexible member and the second flexible member are composed of an absorbing material for absorbing moisture formed on the user's face.

15. The interface of claim 13, wherein the first flexible member and the second flexible member are composed of a non-allergenic material.

16. The interface of claim 13, wherein the first flexible member and the second flexible member are composed of one of a washable material and a disposable material.

17. The interface of claim 13, wherein an aroma-dispersing substance is disposed on the interface for dispersing at least one of aromatic scents and medicinal vapors.

18. The interface of claim 13, wherein at least one of the first flexible member and the second flexible member is composed of a cushioning material.

19. The interface of claim 13, wherein the interface further comprises a coupling mechanism for coupling the interface with the user's face.

20. The interface of claim 13, wherein the coupling mechanism comprises a peel-off cover and an adhesive layer, wherein the adhesive layer is disposed on the second flexible member and the peel-off cover is removably coupled to the adhesive layer.
